(19) United States
(12) Patent Application Publication
(10) Pub. No.: US 2004/0171017 A1
(43) Pub. Date: Sep. 2, 2004

(54) Method to distribute liquids containing molecules in solution and to deposit said molecules on solid supports, and relative device

(76) Inventor: Giuseppe Firrao, Udine (IT)

Correspondence Address: STEVENS DAVIS MILLER & MOSHER, LLP, 1615 L STREET, NW, SUITE 850, WASHINGTON, DC, 20036, US

(21) Appl. No.: 10/482,372

(22) PCT Filed: Jul. 2, 2002
(86) PCT No.: PCT/IB02/02535

(30) Foreign Application Priority Data
Jul. 2, 2001 (IT) UDO01A00114

(51) Int. Cl. C12Q 1/68

(52) U.S. Cl. 435/6; 436/518

(57) ABSTRACT

Method and device to distribute at least a sample of liquid containing molecules in solution, and to deposit and bind the molecules to solid supports. The method provides to use at least two supports (12; 112) which have been previously equipped with at least a through hole (11) and to arrange them one on top of the other so that the respective holes (11) are aligned so as to define a containing channel (13). The method then provides to distribute the sample of liquid inside the containing channel (13) using a surface inside the holes (11) as a site for depositing, localizing and binding the molecules in solution in the sample of liquid.

### FIELD OF THE INVENTION

The invention concerns a method to transfer liquids containing molecules in solution, and to deposit the latter in an orderly manner on a solid support, for use in immunological tests or for testing the hybridization of nucleic acids, or in general testing the interaction of molecules for the purposes of diagnosis and research.

The invention also concerns devices suitable to achieve this method.

The invention is applied particularly, though not exclusively, for molecules consisting of polymers of biological interest, hereafter referred to generally as biomolecules.

### BACKGROUND OF THE INVENTION

There are known numerous systems for depositing samples of molecules and biomolecules in orderly manner.

The technology is particularly developed for nucleic acids (see for example: P.O. Brown and D. Botstein, 1999, “Exploring the new world of the genome with DNA microarrays”—Nature Genetics. Supplement 21: 33-37), but is applied also for proteins and other types of biomolecules (G. MacBeath, S. L. Schreiber, 2000, “Printing proteins as microarrays for high-throughput function determination”, Science 289: 1760-1763).

In its simplest form, deposition is achieved with a system to guide the liquids and a membrane made of porous material, typically nitro-cellulose or nylon. This system is known as “dot-blot” if the guide system is formed by channels with a circular section, or “slot-blot”, when the section of the channels is oval.

In the dot-blot system, the guide system consists of a holed plate, made of acrylic material, under which the porous membrane is placed. By exerting a slight depression under the membrane with a vacuum pump system, a solution containing biomolecules which is applied on the membrane through the hole is induced to pass through the membrane itself. This acts as a filter, retaining the biomolecules in its meshes in correspondence with the surface defined by the aperture of the hole. Afterwards, the biomolecules are irreversibly bound to the substrate with a physical treatment of the membrane.

The deposition of the final product will therefore depend on the structure of the guide plate: the standard format consists of 96 points ordered in a matrix of 12 by 8 in a space of about 75×110 mm.

The traditional use of this system entails that the samples being tested (called “targets”) are immobilized on the solid support. They are tested by means of hybridization (or other type of molecular interaction) with a probe biomolecule with pre-determined characteristics (called a “probe”), marked so as to allow the subsequent determination of the recognized samples.

More recently, a variant of the system described above has become more used; in this variant it is not the samples to be analyzed which are immobilized, but the different probes, which are subsequently made to interact with a sample, suitably marked previously, to be analyzed. In this context, the solid support comprising the various “probes” in orderly arrangement is called an “array”. In order to analyze and compare various samples it is necessary to provide arrays which are identical to each other, one for every sample to be analyzed.

Principally, in applications with nucleic acids, the use of arrays comprises the genetic analysis of polymorphisms for diagnostic purposes, the examination of the genic expression; in applications for proteins, the study of the protein-protein and the antigene-antibody interactions; in applications concerning synthetic molecules, the screening of synthesis products.

Arrays can be produced using the same technique as that described for the “dot-blot”, that is, by filtration, assisted by a vacuum, through a porous membrane. This procedure is called “reverse dot-blot” and the arrays thus produced are generally called “macroarrays”.

In this case, however, the density of the points is not high, and only a very limited number of arrays can be produced simultaneously, since every membrane must be mounted individually on the guide and vacuum system. Systems have been developed which do not need a vacuum, but the reproducibility has not proved high.

Recently, scientists have begun to explore the possibility of obtaining higher element densities by incorporating oligonucleotides in a polyacrylamide gel deposited on glass. More recently a technology has been developed to produce high density arrays, called “microarrays”, using non-porous support materials.

Microarrays normally consist of glass supports (such as microscope slides) or plastic material, suitably treated, on which small quantities of solutions containing biopolymers, mainly nucleic acids or proteins, are deposited in an orderly manner. The support surfaces and/or the biopolymers, according to the protocol, are previously treated so as to react and cause the biomolecules to bind to the support.

To maintain limited dimensions, the density of the grid points must be high, and this is obtained by depositing very small quantities of solution of nucleic acids precisely. Typically, a microarray is produced by depositing drops of solution of about 5-10 nanolitres (nl) at a distance of 100-300 μm from each other.

This operation is carried out by a high-precision robotic instrument which picks up an amount of mother solution and distributes it by depositing it on each of the supports in the determinate position.

Since the precision must be very high and the volumes treated are extremely small, the device used to deposit the liquid is extremely important. Various construction methods have been applied, based on different physical principles, in order to obtain precise and reproducible deposits of very small volumes; the systems mainly divide into those which act through the contact of the instrument with the substrate and those which spray the sample onto the substrate without entering into contact therewith.

For examples of such implementations, reference should be made, for example, to S. Granjeaud, F. Bertucci, B. R. Jordan, 1999, “Expression profiling: DNA arrays in many guises” BioEssays 21: 781-790.

In a different implementation, the biomolecules are deposited on and bound not to the surface of plane elements, but to the inner surface of holes made for this purpose on the support or on the inner surface of a porous material. Subsequently, when the assay for which the array has been prepared is carried out, the solution to be tested is forced to pass through the hole or through the porosity of the material. This experimental solution, sometimes called “flow through”, allows to improve the kinetics of the reaction thanks to the advantageous ratio between the exposed surface of the array and the volume of the solution.

Irrespective of the type of mechanism used to deposit the samples, the technology described and currently prevalent provides that the distributor element visits all the points of all the arrays individually.

The number of movements which the mechanical arm of the robot has to make is therefore very high and, since it has to be completed within a time compatible with the efficiency of the process and to prevent any degradation of the biomolecules, it does not normally permit to obtain, even with a high speed machine, more than about a hundred arrays at the same time, each one consisting of 50÷10,000 points.

Systems and methods for the distribution of molecules in a parallel manner have been exploited for other purposes in different field of biology. For example, U.S. Pat No. 6,168,914 describes a method for combinatorial chemical synthesis which uses a parallel approach. However, due to the relatively low densities which can be attained, systems such as U.S. Pat. No. 6,168,914 cannot be used in the preparation of complex arrays of biomolecules for efficient use in the assay of molecular interaction as it was above described.

That patent teaches to use a plurality of middle plates which are adapted to receive interleaving sheets of membrane. The distance of 2 mm or more between the holes in the plates provided in US '914 is too large for the application in the field of preparation of arrays of molecules and with shorter distances the pressure required to ensure the sealing would reach a value which is impractical for routine use.

However, a few suitable methods are known in the art as discussed below.

A parallel system has been developed (D. I. Stimpson, P. W. Cooley, S. M. Knepper and D. B. Wallace, 1998, “Parallel production of oligonucleotide arrays using membranes and reagent jet printing” Biotechniques 25: 886-890) to produce arrays of chemical compounds. In this procedure, sticks or sheets of porous material are impregnated with the various chemical compounds, then assembled so as to form a bundle which is then cut into thin slices corresponding to the arrays.

The patent application EP 0983795 describes an advantageous alternative technology which consists of superimposing sheet-like bodies, making holes on the pile thus obtained and using the space of the holes thus created as a place where the biomolecules (probes) are deposited. With this technology (defined hereafter as parallel), the arrays are not produced by depositing the same sample on all the individual supports with independent operations; on the contrary, a specific sample is deposited on all the supports with a single operation.

In this way, the movements necessary for the machine to complete the deposition of the samples are significantly reduced, and the volume which has to be handled is significantly increased.

However, EP 0983795 not only leaves some problems unsolved with regard to the practical possibility of using the method in an industrial sense (such as for example an efficient way of filling the holes and containing the liquid), but also it has some theoretical limitations which limit its use, preventing particular configurations.

In the first place, with the technology of EP 0983795, it is only possible to create cylindrical holes. Therefore, the biomolecules will find themselves bound to the support on a plane perpendicular to the plane on which the holes have been made. In those cases where the assay for which the array has been prepared takes place with a reaction which develops light radiation in correspondence with the biomolecules (chemiluminescence or electro-chemiluminescence), the determination of the radiation on a plane perpendicular to the surface of the support is not very efficient.

Moreover, the method of EP 0983795 can be applied for as long as the sheet-like bodies consist of a single homogeneous material. However, the same technology is unsuitable for the preparation of arrays where the material is composite, or where it is required the localization of particular materials in specific areas inside the holes.

The present Applicant has studied, devised and embodied this invention to overcome the shortcomings of the state of the art and to obtain other advantages.

### SUMMARY OF THE INVENTION

The invention is set forth and characterized in the main claims, while the dependent claims describe other innovative characteristics of the invention.

The invention refers to a method of parallel distribution for the production of elements containing biomolecules in the inner face of the holes, which method is an alternative to and more versatile than the state of the art.

To be more exact, the invention provides to use supports which have been previously and therefore already equipped with through holes on the surface, to superimpose a desired number of supports (at least two) aligning the relative through holes already present thereon, and to distribute the sample of liquid containing the molecules or biomolecules in solution, using the inner surface of said holes as a site to deposit, localize and bind the samples.

In a preferential embodiment, said supports are of the plate type, substantially plane.

The aligned holes of the superimposed supports therefore form a containing channel which remains open in its upper part and is closed in its lower part by closing means, advantageously made of hydrophobic material.

Two rigid structures with appropriate holes, respectively located above the upper support and below the closing means, keep the whole aligned and securely closed. For the rest of the description, this whole shall be called “distribution sandwich”.

One embodiment of the invention provides the application of a vacuum pump from below and through the closing means in order to ensure the perfect filling of the relative containing channel with a liquid solution containing the biomolecules; thanks to the vacuum application, the air passes through the closing means and the liquid forms a perfect column in the relative containing channel. When the channel is full, the vacuum application is suspended and the channel is sealed to allow the biomolecules to bind to the substrate.

When the biomolecules are stably bound to the walls of the holes, the supports are separated from each other so that each of them has a specific sample, associated with the wall of the relative hole, of biomolecule to be used independently for immunological tests, tests for the hybridization of nucleic acids, or more generally for diagnosis and research purposes.

By using a number of supports which can vary in a substantially unlimited manner, it can be seen how the invention allows to form, with limited liquid transfer actions, a potentially very high number of identical, and even complex, arrays.

In a preferential embodiment, the holed supports are made with the photolithographic techniques typically used in the industrial production of printed circuit boards for electronic devices.

In this preferential embodiment, firstly a central layer of the support is made of metal and/or electrically conductive material; holes are made therein by means of isotropic chemical etching which, at the end of the process, have the shape of a truncated cone.

In the same preferential embodiment, every individual support consists of said central conductive layer, lined on both its upper and lower face by a separating layer, made for example of plastic material.

In a preferential embodiment, the surface of the holes can be coated, only on the central layer, with a different material (active layer).

When the supports are assembled and aligned in the “distribution sandwich”, the central layer of the supports can be electrically connected with an outside electric circuit. When the biomolecules are introduced into the channel and an electrical potential is applied between the supports, one with respect to the other, or between the supports and a further electrode, the biomolecules will covalently bind to the supports by anodic oxidation dependent binding.

There is a wide variety of materials that can be used to make the supports, of methods suitable to produce the binding of the biomolecules to said supports, and of shapes of the inner holes which can advantageously be implemented in this context.

The central layer can consist of any material with high electric conductivity, either metal or plastic. In the first case the central layer will be produced by means of photolithography-etching, in the second by means of molding, according to conventional technology.

The separating layer (positioned both above and below the central layer) can consist of any material with low electric conductivity, such as (but not only) glass, silicon oxide or nitride, and especially plastic material, for example various photopolymers. Depending on the material, the separating layer can be produced with photo-lithographic processes, coating processes, oxidizing processes, molding processes or gluing processes, according to conventional technology.

The active layer can consist of any material which can work as an electrode, either metal or plastic, for example graphite, glassy carbon, gold, platinum, silver, various synthetic materials. They may be localized inside the holes either by means of the traditional systems used industrially to make contacts between the two faces of printed circuits (“through hole processing”), or by means of coating systems, by electrocoating, electroplating, sputtering, galvanic deposition, vacuum deposition.

According to the process used, it may be useful to proceed first to lay down the separating layers or first to coat the inner surface of the holes.

The process may require the use of additional processes such as masking and mask removal, without substantially modifying the characteristics of the invention. The supports may consist of a greater or lesser number of layers without substantially modifying the characteristics of the invention. In all cases, the present invention does not refer specifically to the process of making the supports, which can be achieved by adapting state of the art techniques, of which there are innumerable variants, but rather to the specific use of said supports thus conceived in order to make and use arrays of biomolecules.

As we have said, when the supports are assembled and aligned in the “distribution sandwich”, the central conductive layer of the supports can be electrically connected to an external electric circuit.

More complex constructional modalities can be implemented so that supports can be made with conductive materials and separator suitably localized so that it is possible to establish specific electric contacts for every individual hole of the support. In this way every channel or cell constituted by the hole when the biomolecules are deposited, or at the moment of the assay can be addressed individually.

The shape of the inner surface of the hole can also be different.

In a preferential embodiment, as we have said, the holes of the central layer are shaped like a truncated cone, while the corresponding apertures in the separating layers are cylindrical in shape; the upper layer and the lower layer advantageously are of different diameter.

In alternative embodiments, the central layer has its space occupied by different shapes, other than cylindrical, for example trapezoid, triangular or otherwise, while the separating layers have cylindrical shaped holes. Other solutions are possible, but substantially finalized to obtain a final configuration in which the central conductive layers of the different supports (i) are not in direct contact with each other, since they are separated by the respective separating layer, and (ii) have a surface which can be wet by the liquid introduced into the channel constituted by the superimposition of the different supports.

The holes of numerous superimposed independent supports constitute the same number of channels, each of which can be filled with a single dosage of liquid. The following advantages are therefore obtained:

the amount of liquid which has to be managed by the liquid distribution system in a single operation is not the amount needed to serve a single element of a single support, but that corresponding to a single element multiplied by the number of supports.

In the case where the arrays are produced on material with a thickness of 0.25 mm with holes with a radius of 0.3 mm, each hole of each support has a volume of about 70 nl.

In the production of 100÷1000 arrays, the volumes of the individual transfers of liquids will be equal to 7÷70 μl, that is, volumes which can be treated with manual instruments or with automatic instruments of a conventional type, characterized by a good level of efficiency.

The number of movements which a machine has to make to serve all the points is not equal to the number of the samples to be transferred (elements) multiplied by the number of supports, but to the number of elements alone. This characteristic makes it extremely economical to produce arrays with few elements. For example the production of 1000 arrays of 500 elements requires 500 movements to distribute the liquid, instead of 500,000; the production of 100 arrays of 30 elements requires 30 movements and can be easily performed with manual distribution pipettes.

The dosage which comes into contact with the binding surface does not depend on the volume transferred but on the volume of the hole. Thus, each of the supports receives the same dosage even when the distributing machine is not very precise.

Since the upper surface of the pile of supports is not affected by the interaction between support and biomolecule, it is possible to put on top of the supports a suitable funnel-shaped device suitable to concentrate the liquid in the exact points of the holes, offering at the same time a wider surface for the machine to deposit the sample. In this way, the precision needed to deposit the sample is reduced, and this can therefore be done manually or with the aid of a conventional robot equipped with pipettes (for example the more economical models of the Biomek® series by Beckman Coulter, Inc.) even for those applications which require a high density of the elements.

Since the deposition of the samples of liquid is not planar, the phenomena of reciprocal interference are reduced to a minimum (for example when the signal is detected with chemiluminescent or electro-chemiluminescent means).

The determination of the final product is rendered less critical when the product is developed inside a hole. For example, in the case of nucleic acids, the arrays can be hybridized with a probe, marked with an electro-chemiluminescent or electrically active group. During the reaction which constitutes the assay (for example during hybridization), or at the end thereof, the individual holes can constitute independent reaction cells which, under the action of an electric field, produce a diagnostic reaction, such as for example the generation of light radiation. With this method, we limit the interference between the emission of light of one element and that of the adjacent elements and we obtain a better spatial resolution than it is possible to obtain when the elements emitting light lie on one plane.

Due to the disposition and entity of the volumes which are characteristic of this invention, it lends itself to being integrated into a system which comprises the transfer of liquids according to an analogous principle.

Due to these characteristics, the present invention lends itself to being integrated into signal detection systems which couple the use of chemiluminescent or electro-chemiluminescent methods with solid-state signal determination systems, allowing to develop integrated systems which are economical but with very high performance.

### BRIEF DESCRIPTION OF THE DRAWINGS

These and other characteristics of the invention will be clear from the following description of a preferential form of embodiment, given as a non-restrictive example with reference to the attached drawings wherein:

FIG. 1a shows the basic principle used in the method according to the invention in a schematic and exploded view;

FIGS. 1b and 1c show respectively the superimposed supports of FIG. 1a and the detail of a channel for the liquid containing the biomolecules consisting of the aligned holes of said supports;

FIGS. 1d and 1e show the principle for achieving the invention, in an exploded view and in a partly assembled condition;

FIG. 2a shows a support in correspondence with a hole in a schematic and cutaway view;

FIG. 2b shows a section of a combination of superimposed supports in correspondence with the channel formed by the holes;

FIGS. 2c, 2d, 2e and 2f show alternative configurations of the holes in the supports with which the invention can be embodied;

FIGS. 3a and 3b show a form of embodiment of a device which achieves the method according to the invention in an exploded view and in a partly assembled condition;

FIG. 4 shows a form of embodiment of an assembly element for a part of the device shown in FIGS. 3a and 3b in an exploded view;

FIG. 5 shows a possible application of the device according to the invention;

FIG. 6 shows a form of embodiment of a closing element of the device shown in FIG. 5;

FIG. 7 shows a form of embodiment of an assembly element for a part of the device shown in FIG. 5 in an exploded view.

### DETAILED DESCRIPTION OF SOME PREFERENTIAL FORMS OF EMBODIMENT OF THE INVENTION

Hereafter, we shall describe in detail some preferential forms of embodiment of a device and its main components which achieve the method according to the invention. Other configurations are possible, in addition to and as variants of those described, for example with different densities, channel diameter and number of points.

The mechanical embodiment can also differ, at least with regard to the distribution of the samples of liquids; for example, a variant may provide a system to move the structure which contains the samples, instead of a movement with a movable plate above the containing structure, as in the embodiment shown.

The number of positions of the movable plate with respect to the structure which contains the samples can also be varied, allowing to obtain different point densities.

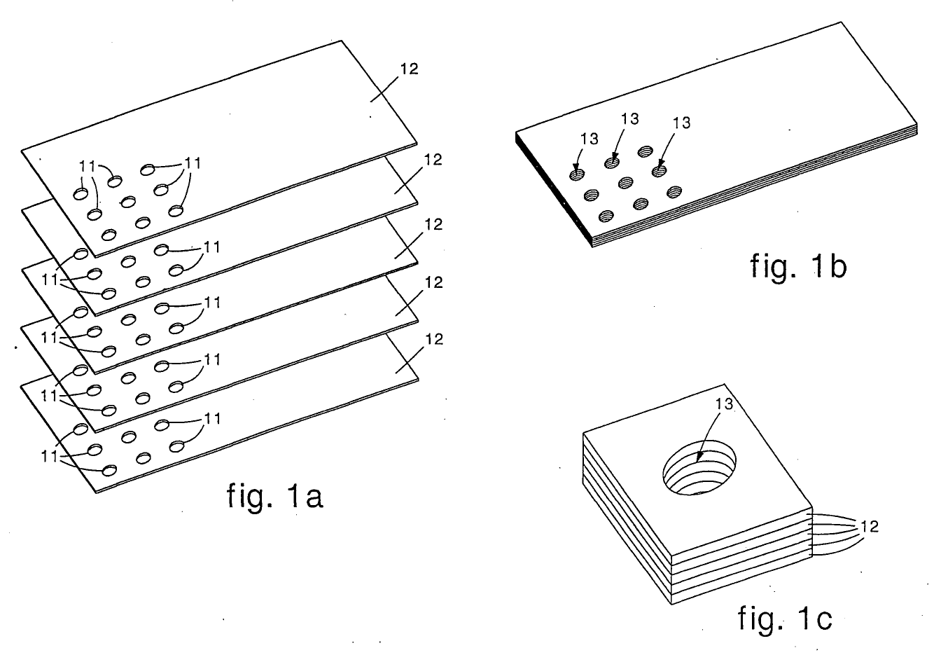

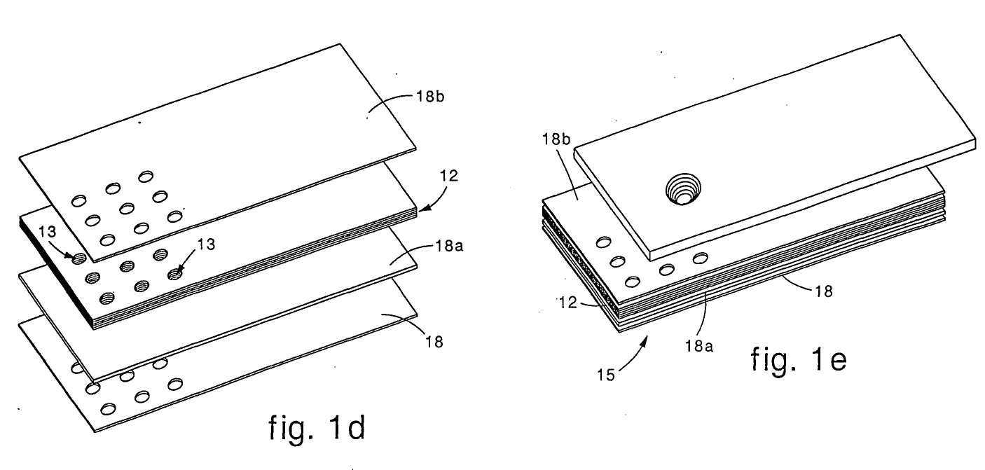

FIG. 1 shows the principle on which the invention is based, that is, the use of the inner area of holes 11 previously made on supports 12 for depositing samples of liquids containing biomolecules in solution.

The supports 12, seen separated in FIG. 1a, already equipped with the respective holes 11, are superimposed and aligned with each other, as shown in FIG. 1b, so as to constitute channels 13 into each of which the liquid can be introduced (FIG. 1c). The inner surface of said channels 13 constitutes the physical support to which the biomolecules are bound.

FIGS. 1d and 1e show how the principle of the invention can be achieved by means of a plurality of supports 12 one on top of the other, with at least a porous membrane of the hydrophobic type 18a interposed, an upper plate 18b and a lower plate 18 to close the whole and make it rigid.

If necessary, an upper plate for distributing the samples of liquids, with a funnel-shaped discharge hole, is associated with said elements, as will be explained later.

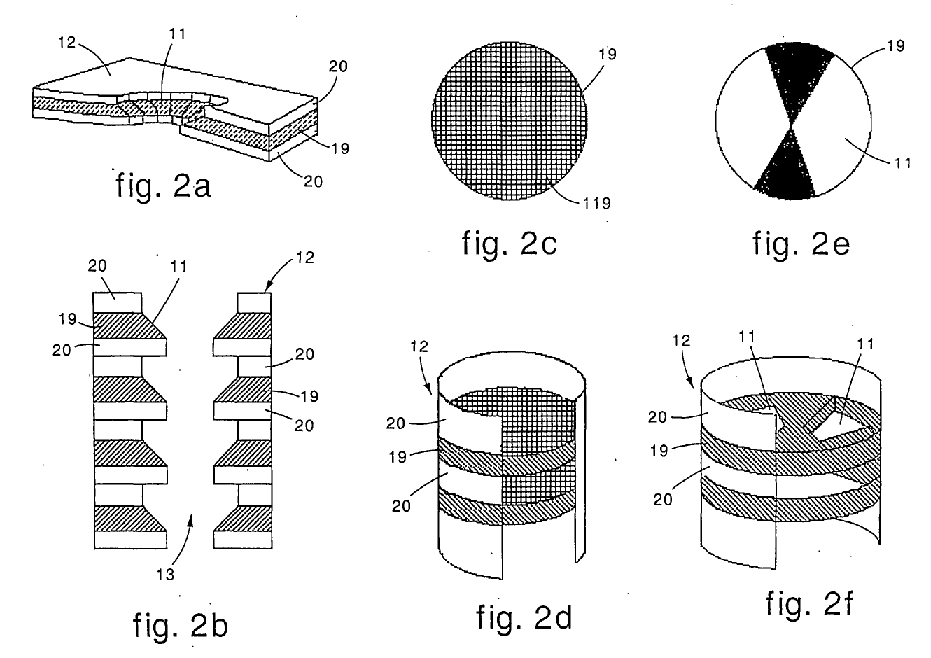

FIG. 2a shows, in a cutaway view, a support 12 in correspondence with a hole 11. As can be seen, every support 12 is formed by a central layer 19 coated with separating layers 20. The shape of the hole 11 is not cylindrical, since it is shaped like a truncated cone, but only in the central conductive layer 19. The central layer 19, but only on the inner surface of the hole 11, is coated with an active layer of about 1 μm of “Electrodag PR-406”, a carbon polymer thick film ink produced by Acheson Colloiden B. V. (Holland). FIG. 2b shows the section of the channel resulting from the superimposition of various supports 12 in correspondence with the channel 13.

FIGS. 2c-2e show alternative forms of the holes 11 made in each of the supports 12, 112.

In the embodiment shown in FIGS. 2c and 2d, the central layer 19 is made in the shape of a grid 119 which partly obstructs the holes 11 made in the upper and lower separating layers 20. The grid 119 constitutes the surface on which the molecules in solution in said sample of liquid are deposited, localized and bound.

In the embodiment shown in FIGS. 2e and 2f, the holes 11 made in the central layer 19 are substantially trapezoid in shape.

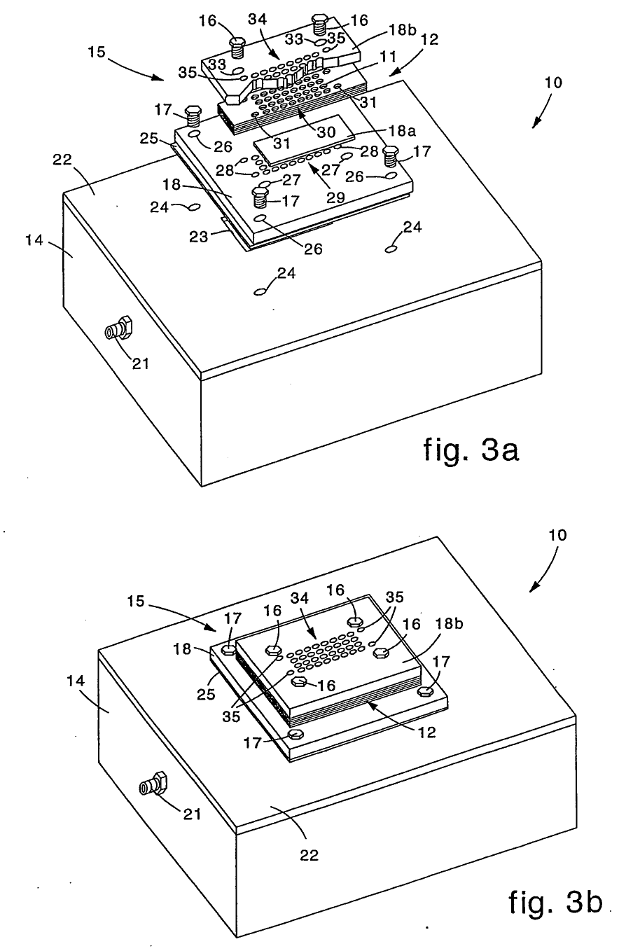

With reference to FIGS. 3a and 3b, a device 10 according to the invention, shown in an exploded view in FIG. 3a and in an assembled condition in FIG. 3b, comprises a vacuum chamber 14, above which a so-called “distribution sandwich” 15 rests, with a sealed coupling.

The “distribution sandwich” 15 is a rigid block consisting of a plurality of components held tightly together by means of sealing screws 16, and is connected to the vacuum chamber 14 by means of closing screws 17. To be more exact, the “distribution sandwich” 15 consists of the lower plate 18, the porous membrane 18a, the supports 12, stacked and aligned together, and the upper plate 18b, which is also holed.

With regard to the detailed description, the vacuum chamber 14 consists of a block, for example made of aluminium, parallelepiped in shape. The block is excavated so as to obtain a chamber, with walls for example of about 10 mm in thickness, open on the upper side; on one wall said chamber has another small circular aperture 21 through which a vacuum pump is connected to create a vacuum in the chamber 14.

The upper part of the chamber 14 is closed by a slab 22, for example made of stainless steel, on which an aperture 23, like a rectangular window, and four holes 24 able to receive the closing screws 17 are made.

Above said aperture 23 a seal 25 is positioned to ensure the vacuum holds when the “distribution sandwich” 15 is operated.

As we have seen above, the “distribution sandwich” 15 consists of elements which are held in close contact with each other.

The lower base plate 18, for example made of stainless steel, has four outer holes 26 to connect with the vacuum chamber 14, by means of the closing screws 17, and four inner holes 27 to connect with the other elements of the “distribution sandwich” 15, by means of the sealing screws 16.

On said base plate 18 another four inner holes 28 are made to assemble and align with the other elements of the “distribution sandwich” 15, by using an assembly plate 36, as will be seen later. The plate 18 has thirty-two holes 29 constituting a four by eight grid; in the preferential embodiment, said holes 29 have a diameter of about 1 mm and are distanced from each other by about 1.5 mm.

They define the loading points of the samples of liquid with biomolecules in solution for the formation of the individual arrays.

Above the holes 29 the porous membrane 18a is placed, which in the preferential embodiment is made of Teflon (PTFE) of the Mitex™ type produced by Millipore Corp.

In a preferential embodiment, the supports 12 for the arrays consist of a central layer 19 made of copper, wherein only the inner surface of the holes 11 is coated with a layer of about 10 μm of “Electrodag PR-406”, a carbon polymer thick film ink produced by Acheson Colloiden B. V., and of upper and lower separating layers 20 made of photopolymeric resin Rigilon®. Such supports vary in number according to necessity, and preferably have a thickness comprised between 0.15 and 0.45 mm, advantageously 0.25 mm.

The number of said supports, in normal applications, can vary from 10 to 500, but it is possible to hypothesize uses which provide 5-10,000 superimposed supports without particular limits.

Thirty-two holes 11, in this case, are made on said supports 12, mating with the holes 29 on the base plate 18, about 1 mm in diameter and distanced from each other by about 1.5 mm, which correspond to the loading points of the samples. The holes 11 constitute a four by eight grid, denoted generally by the reference number 30.

The supports 12 also include, in proximity with the perimeter, four holes 31 to align and assemble with the other elements of the “distribution sandwich” 15.

The “distribution sandwich” 15 also comprises an upper plate 18b, advantageously made of stainless steel, arranged above the supports 12; said upper plate 18b, in proximity with the perimeter, has four holes 33 to connect with the other elements of the “distribution sandwich” 15 by means of the sealing screws 16. The space of the holes 34, cylindrical in shape, is coated with platinum.

The upper plate 18b also has thirty-two holes 34, constituting a four by eight grid, in correspondence with the holes 29 of the base plate 18 and the holes 11 of the supports 12.

In correspondence with the perimeter, the upper plate 18b also has four holes 35 to assemble the “sandwich” 15. The holes 35 and the upper and lower face of the plate 18b are coated with Teflon, so that also after assembly the plate 18b is electrically insulated from the rest of the “sandwich” 15.

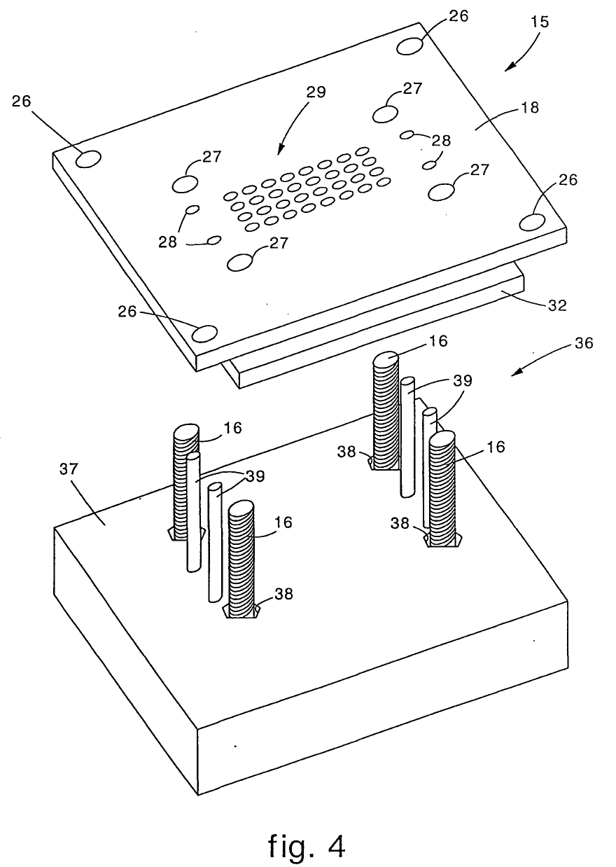

The assembly is achieved by using an assembly plate 36, shown in FIG. 4. It comprises a plate 37, preferably made of acrylic material, on which four seatings 38 are made for inserting the sealing screws 16. Four guides 39 are also made on said plate 37, consisting of steel cylinders advantageously with a diameter of about 1 mm.

The “distribution sandwich” 15 is assembled by inserting the various elements, respectively lower plate 18, supports 12 and upper plate 18b, upside down on the guides 39, by means of the respective holes 28, 31 and 35.

To be more exact, a first assembly step provides that the sealing screws 16 are inserted into the respective seatings 38 on the plate 37; then, the upper plate 18b and the supports 12 are inserted upside down, using the guides 39.

Then, a piece of membrane 18a is cut to size and positioned above. Finally, the lower base plate 18 is inserted, the “sandwich” 15 thus obtained is turned over and constrained, by tightening the sealing screws 16.

The “sandwich” 15 is removed from the assembly plate 36 and put in a furnace to allow the supports 12 to adhere perfectly to each other and to the membrane 18a. The sealing screws 16 are then further tightened, and exert clamping pressure on the “sandwich” 15 while the latter is still hot, so as to further promote the perfect adhesion of the supports 12 to each other and to the membrane 18a. The “sandwich” 15 is then mounted on the vacuum chamber 14.

Then, the samples to be deposited on the supports 12 are prepared.

The samples can be biomolecules of various types. In a preferential application of the device 10 according to the invention, the samples consist of fragments of nucleic acids modified by a primary amine at position 5′.

In order to load the samples, the vacuum chamber 14 is connected to a vacuum pump. The first sample to be deposited is picked up with a pipette in an amount determined by the number of arrays which have to be prepared, that is, 0.785 microlitres for every support 12, plus a slight excess (about 3 microlitres). If 20 arrays are prepared, about 19 microlitres are picked up, and the tip of the pipette is brought near to the hole corresponding to the first position. The tip is positioned vertically, inserted by about a millimetre into the hole of the upper plate 18b and kept in position so as to allow the liquid contained in the tip to flow into the apparatus, drawn by the depression therein. The liquid thus fills the channel made up by the holes in the supports 12 and in the upper plate. The tip is replaced and the process repeated for all the samples.

At the end of loading the upper plate 18b is coated with an adhesive sheet to prevent evaporation. Then, an electrical potential is applied between the body of the device 10 and the plate 18b.

The “sandwich” 15 is then taken apart. The supports 12 are separated from each other, the polyethylene or polymer film separating them is eliminated and, after a possible washing process, they are ready to be used in hybridization experiments of nucleic acids.

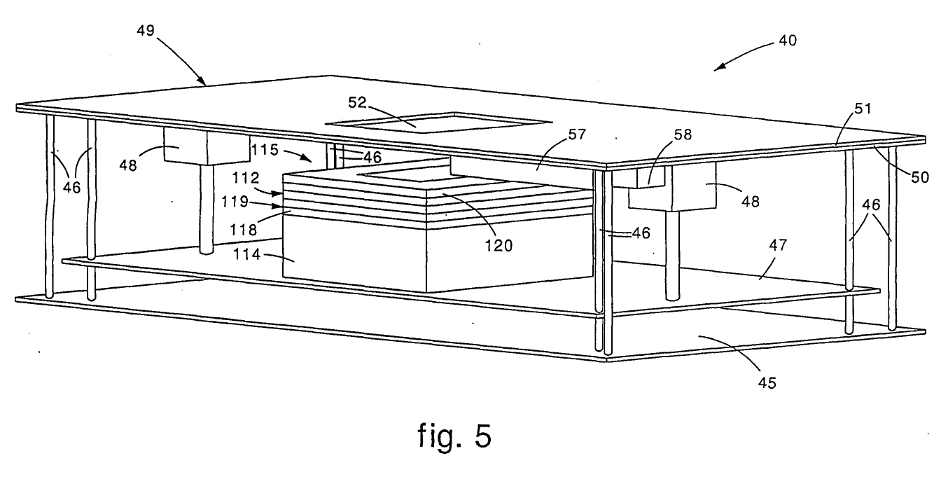

FIG. 5 shows a device 40 able to perform automatically the deposition method previously described with a manual application. We shall now describe the parts of said device 40 which differ from those already described with reference to FIGS. 3a and 3b.

The structure for containing the samples, conceptually similar to the one described above, is positioned on a plane movable on the axis z, mounted in a supporting structure on the upper part of which there is a plate movable on the axes x and y.

Said device 40 comprises a vacuum chamber 114, similar to the one described previously, but differs in that it has a greater size of the window-type aperture 23 made on the upper part.

The “distribution sandwich” 115 consists of a base plate 118, a membrane 119, a plurality of supports 112 for the arrays, preferentially from 10 to 300 in number, according to necessity, and an upper plate 120.

In this case, both the base plate 118 and the supports 112 and the upper plate 120 have external holes for connection with the vacuum chamber 114, ten internal holes for connection, by means of sealing screws, with the other elements of the “distribution sandwich” 115, and ten internal holes for alignment with the other elements of the “distribution sandwich” by means of the guides of the assembly plate 136.

There are also 6144 holes made on said elements 118, 112 and 120, which holes constitute a 64×96 grid, with a diameter of about 0.7 mm and distanced from each other by about 1.1 mm, which correspond to the loading points for the samples.

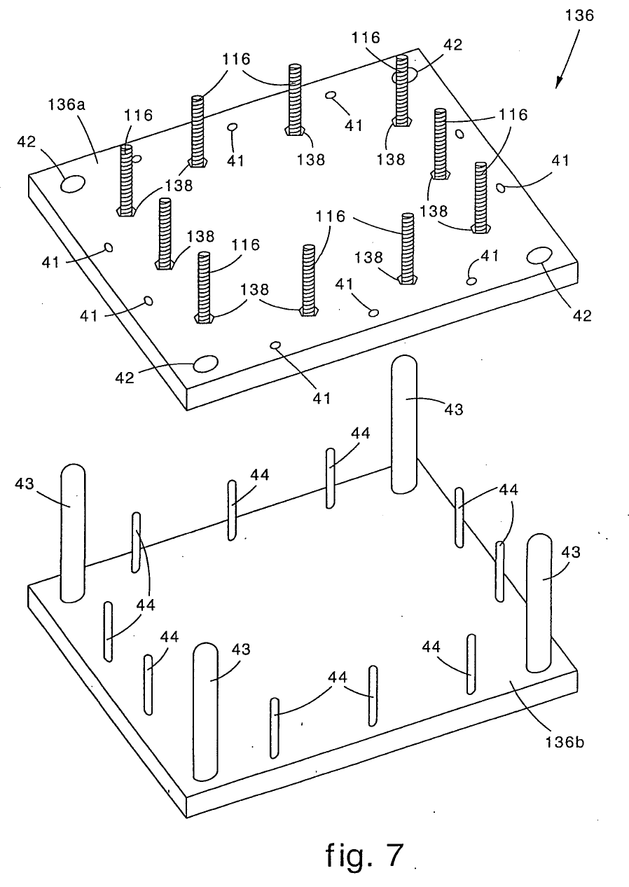

In this case, the “distribution sandwich” 115 is mounted with the aid of two assembly plates 136a and 136b, advantageously made of acrylic material and shown in FIG. 7.

On the first plate 136a there are ten seatings 138 for the sealing screws 116, ten holes 41 with a lesser diameter and four holes 42 of a greater diameter for the first alignment with the second plate 136b.

Said second plate 136b has four wide guides 43, substantially in correspondence with the corners, for the first alignment with the first plate 136a, and ten thin guides 44, arranged along the sides.

The “distribution sandwich” 115 is prepared on the first plate 136a by inserting, by means of the guides 43 and 44, the various, overturned elements, starting from the upper plate 120. When assembly is complete, the second plate 136b is inserted from below to obtain the perfect alignment of the holes. Then the sealing screws 116 are tightened and the assembly plates 136a and 136b removed and taken away.

The device 40 comprises a support and orientation structure consisting of a base plane 45, advantageously made of aluminium; in a preferential embodiment, said base plane 45 is suitable to house elements for connection with a computer, pneumatic valves and other means necessary for the functioning, management and control of the device 40.

Supporting shafts 46 are mounted on the base plane 45 substantially in correspondence with the vertices; an intermediate plane 47, also advantageously made of aluminium, is able to slide on the supporting shafts 46, thanks to the action of two actuators 48, in this case pneumatic cylinders, the fixed part of which is mounted on a covering plane 49.

Said covering plane 49 consists of two plates, respectively lower 50 and upper 51, advantageously made of aluminium, including respective windows where a movable plate 52 is positioned.

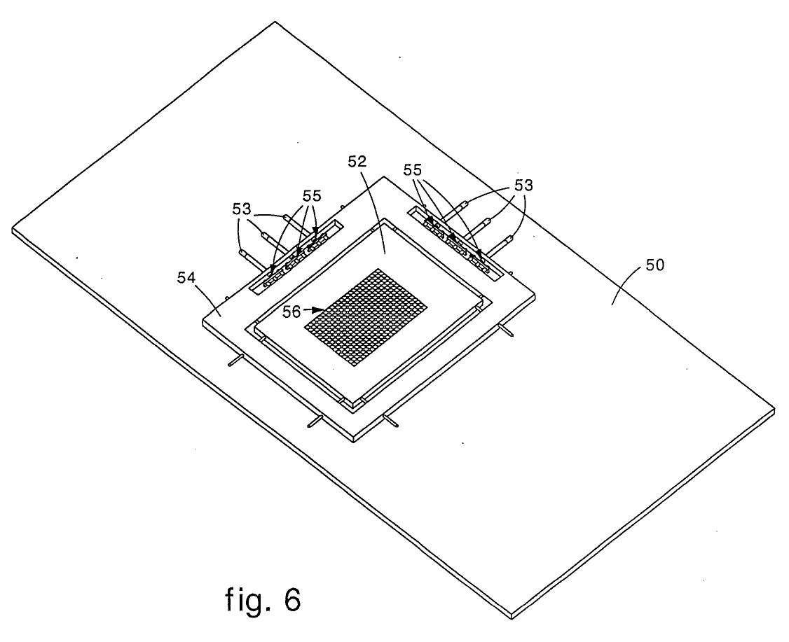

The lower plate 50 contains the system to move the movable plate 52 which, as shown in FIG. 6, comprises six pneumatic cylinders 53, attached to the lower plate 50 by means of a frame 54, which drive mating thrust elements 55 of the movable plate 52.

Said thrust elements 55, also preferentially made of aluminium, are able to slide on rails made in the frame 54 following the thrust caused by the extension of the cylinders 53, causing the movable plate 52 to move.

Suitable calibration screws, positioned in their rear part, function as end-of-travel elements for the thrust elements 55.

There are also mechanical elements which encourage the return of the movable plate 52 and the thrust elements 55 when the pneumatic cylinders 53 return to rest.

The movable plate 52 is advantageously made of aluminium and, in this case, has 384 holes 56 arranged regularly in 24 rows of 16 elements. The holes 56 are funnel-shaped, that is, like a cylinder surmounted by an overturned cone.

The device 40 also comprises a washing plate 57, advantageously made of acrylic material and comprising a thin chamber connected to a vacuum system. It can be moved by means of the action of a pneumatic piston 58 so as to be inserted between the movable plate 52 and the structure for containing the samples.

The device 40 also comprises at least a compressor to feed the pneumatic cylinders 48, 53, 58, pneumatic valves and a computer with interface and program to control them.

The principle for loading the samples is similar to that described previously for the manual apparatus. It differs therefrom mainly in the greater density and number of holes on the upper plate 51, which would make a manual loading long and problematic.

In this device, in fact, the upper part of the “distribution sandwich” 115 is pressed against a plate, that is, the movable plate 52, which has funnel-shaped holes 56 equal in number to one sixteenth of the number of holes in the upper plate 51.

The position of the movable plate 52 can be determined accurately by the movement of said thrust elements 55 driven by the mating pneumatic cylinders 53, and is such as to make the lower part of said funnel-shaped holes 56 correspond perfectly with the channels of the “distribution sandwich” 115. Obviously, when the movable plate 52 is in a certain position, the funnel-shaped holes 56 will be simultaneously in communication with one sixteenth (384) of the holes of the upper plate 51 of the “sandwich” 115, while the others will be closed.

The funnel-shaped holes 56 have a lower diameter substantially equal to that of the holes in the upper plate 51 against which they are pressed (around 0.7 mm, in the preferential embodiment of the invention), while in the upper part their diameter is about 3.5 mm.

Thanks to this arrangement it is possible to fill the 0.7 mm channels of the “distribution sandwich” 115 through the upper aperture of the funnel-shaped holes 56, which has a much larger diameter and therefore does not require a great level of accuracy for this filling operation. The volume inside the funnel is sufficient to contain the excess liquid which can be created temporarily when the liquid of the solution to be loaded emerges from the tip of the pipette and enters the channels, sucked in by the vacuum.

The movable plate 52 has 384 holes 56, positioned exactly like the conventional plates for storing samples with 384 pits (for example those produced by Dynex Technologies). Using a multiple pipette distributor with 96 channels it is therefore possible, with only four movements, to fill the channels corresponding to 384 samples which have been first arranged in plates for 384 or 96 samples.

When the first 384 samples have been loaded, the sliding intermediate plane 47 is lowered. In this way the funnels of the movable plate 52 can be washed; the washing plate 57 is inserted by pneumatic movement between the “distribution sandwich” 115 and the movable plate 52; the sliding intermediate plane 47 is raised slightly, allowing a perfect contact between the parts and the vacuum seal which is exerted to eliminate the washing liquid.

The sliding intermediate plane 47 is then lowered again, the washing plate 57 withdrawn and the movable plate 52 is put in another position by the movement of the thrust elements 55 driven by the relative pneumatic cylinders 53. This movement is performed so as to make the lower part of the funnel-shaped holes 56 correspond perfectly with 384 channels of the “distribution sandwich” 115, different from those previously loaded.

The process is repeated 16 times so as to fill all the channels of the “distribution sandwich” 115.

The samples may consist, in this case, of synthetic oligonucleotides of deoxiribonucleic acid, marked with biotin and subsequently made to react with streptavidin.

Other applications can provide that the samples consist of fragments of nucleic acids amplified by means of chain reaction of the polymerase (PCR) and marked with biotin-streptavidin, or with another system which will allow the subsequent adsorption of the biomolecules to the solid support.

The “distribution sandwich” 115 is mounted with operations substantially identical to those described previously.

When said “sandwich” 115 has been mounted in a suitable position on the device 40, the program sets the appropriate opening of the pneumatic valves and consequently the movement of the cylinders 53 and of the relative thrust elements 55, so that the movable plate 52 is positioned in one of the sixteen possible configurations.

The program sets the opening of the pneumatic valve which allows the sliding intermediate plane 47 to rise, consequently pressing the upper plate 51 of the “distribution sandwich” 115 against the movable plate 52. The 384 channels of the “distribution sandwich” 115 which are thus connected to the funnel-shaped holes 56 of the movable plate 52 are thus filled, either individually with the aid of a robot (for example one of the Biomek® 2000 series produced by Beckman Coulter Inc.) or in groups of 8-96 with a multichannel pipette distributor.

The program sets the opening of the pneumatic valve which allows the sliding intermediate plane 47 to descend and the washing plate 57 to slide between the movable plate 52 and the upper plate 51 of the “distribution sandwich” 115. The sliding intermediate plane 47 is made to rise so as to press the washing plate 57 against the movable plate 52. The movable plate 52 is filled with water which, sucked in by the vacuum exerted in the washing plate 57, filters through the funnel-shaped holes 56 and washes them.

This cycle is repeated another 15 times, so that all the 16 configurations have been obtained.

It is clear however that modifications and/or additions of parts can be made to the method and device as described heretofore, without departing from the spirit and scope of the invention.

1- (Amended) Method to distribute at least a sample of liquid containing molecules in solution, and to deposit and bind said molecules to solid supports, characterized in that it provides to use at least two supports (12; 112) which
have been previously equipped with at least two through holes (11) spaced but extremely close each other and
have been previously arranged in direct contact one on top of the other
said two supports (12; 112) being aligned so that the respective holes (11) define a containing channel (13), the method further providing to distribute said at least one sample of liquid inside said containing channel (13) using a surface inside said holes (11) as a direct site to deposit, localize and bind the molecules in solution in said sample of liquid,
in which, having terminated the step wherein the molecules in solution are bound to the inner surface of the hole (11) of the support (12; 112), said supports (12; 112) are separated from each other, each defining a collection of. immobilized molecules for use in experiments and tests for diagnosis and research.

2- (New) Method as in any claim hereinbefore, characterized in that said through holes (11) are spaced each other about 1,5 mm or less.

3- Method as in claim 1 or 2, characterized in that the surface on which said molecules in solution in said sample of liquid are deposited, localized and bound forms an angle of less than 90° with the surface of said supports (12; 112) on which said holes (11) have been made.

4- Method as in claim 1 or 2, characterized in that each of said supports (12; 112) consists of a central layer (19) coated on at least one face with at least a separating layer (20) of different material.

5- Method as in claim 4, characterized in that said hole (11) is formed by the separating layers (20) and is partly obstructed at the center by the central layer (19) in the form of a grid which constitutes the surface on which said molecules in solution in said sample of liquid are deposited, localized and bound.

6- Method as in any claim hereinbefore, characterized in that said surface on which said molecules in solution in said sample of liquid are deposited, localized and bound is coated with a material suitable for allowing or improving the binding of said molecules.

7- Method as in claim 6, characterized in that said material is localized exclusively on said central layer (19) and not on said separating layer (20).

8- Method as in claim 1, characterized in that after the molecules have been bound to said inner surface of the holes (11), it provides that said at least two supports (12) are separated from each other to be used in independent manner.

9- Method as in any claim hereinbefore, characterized in that said molecules consist of polymers of biological interest, or biomolecules.

10- Method as in claim 9, characterized in that said biomolecules are nucleic acids.

11- Method as in claim 9, characterized in that said biomolecules are proteins.

12- Method as in claim 9, characterized in that said biomolecules are substituted by functional groups.

13- Method as in claim 1, characterized in that it provides to use at least selective means (18a; 119) arranged below the support (12;112) located at the bottom, to close said containing channel (13) for the sample of liquid defined by the aligned through holes (11) of said at least two supports (12; 112).

14- Method as in claim 13, characterized in that said means (18a; 119) consist of at least an element made of hydrophobic material.

15- Method as in claim 13, characterized in that said means (18a; 119) comprise at least a porous membrane.

16- Method as in claim 13, characterized in that it provides at least a step of applying vacuum at least from below said closing means (18a; 119) to obtain a desired filling of the containing channel (13) defined by said aligned through holes (11) of said at least two supports (12; 112).

17- Method as in claim 1, characterized in that when said containing channel (13) has been filled with said sample of liquid, it provides that said containing channel (13) is sealed to allow the binding of the biomolecules to the inner surfaces of the hole (11) of the respective support (12; 112).

18- Method as in claim 17, characterized in that it provides that, after sealing, the liquid inside said containing channel is subjected to heat, electric or other physical treatment in order to obtain the binding of the biomolecules to the surfaces inside the hole (11) of the respective support (12; 112).

19- Method as in claim 1, characterized in that when said containing channel (13) has been filled with said sample of liquid, it provides to apply an electric potential in order to obtain the binding of the biomolecules to the surfaces inside the hole (11) of the respective support (12; 112).

20- Method as in any claim hereinbefore, characterized in that after the containing channel (13) has been filled, it provides that said supports (12; 112) are arranged in an environment at a controlled temperature to promote the binding of the biomolecules on the inner wall of said through holes (11).

21- Method as in any claim hereinbefore, characterized in that it provides that said containing channels (13) are filled sequentially in manual mode.

22- Method as in any claims from 1 to 20 inclusive, characterized in that it provides that a plurality of said containing channels (13) are filled simultaneously and automatically by means of suitable distributor means (52, 53, 55).

23- Method as in claim 22, characterized in that said simultaneous filling of a plurality of containing channels (13) occurs with a single movement of said distributor means (52, 53, 55).

24- Device to distribute and deposit on solid supports at least a sample of liquid containing molecules in solution, characterized in that it comprises at least two supports (12; 112) which have been already previously equipped with at least two through holes (11) spaced but very close each other, by a distance of 1,5 mm or less, which may be partly obstructed, and arranged one on top of the other so that the respective holes (11) are aligned so as to define a containing channel (13), means (18a; 119) arranged below the support (12; 112) located at the bottom to close said containing channel (13), and means able to distribute said at least one sample of liquid inside said containing channel (13) using a surface inside said holes (11) as a direct site to deposit, localize and bind the molecules in solution in said sample of liquid.

25- Device as in claim 24, characterized in that it comprises means (14; 114) able to create a vacuum associated at least with below said supports (12; 112).

26- Device as in claim 24, characterized in that it comprises at least a lower base plate (18; 118) and at least an upper base plate (18b; 120), said plates (18, 18b; 118, 120) including respective holes (29, 34) in correspondence with the holes (11) made on the supports (12; 112) and able to enclose said superimposed and aligned supports (12) and said closing means (18a) between them.

27- Device as in claim 26, characterized in that said elements (12, 18, 18a, 18b; 112, 118, 119, 120) are able to be constrained one to the other by means of joining means (16; 116), forming in their entirety a rigid block (15; 115) able to be associated at least temporarily to said means (14; 114) able to create a vacuum to distribute the samples of liquid inside the holes (11) of said supports (12; 112).

28- Device as in claim 25, characterized in that said means able to create a vacuum comprise at least a chamber (14; 114) including at least a first aperture (21) for connection with pump means able to create a vacuum and at least a second window-type aperture (23) for positioning said rigid block (15; 115) by means of clamping means (17).

29- Device as in claim 28, characterized in that sealing means (25) are interposed between said second window-type aperture (23) and said rigid block (15; 115).

30- Device as in any claims from 24 to 29 inclusive, characterized in that each of said supports (12; 112) has a thickness comprised between about 0.15 and 0.45 mm.

31- Device as in any claims from 24 to 30 inclusive, characterized in that said through holes (11) made on said supports (12; 112) have a maximum diameter of about 1 mm.

32- Device as in any claims from 27 to 31 inclusive, characterized in that it comprises assembly means (36; 136a, 136b) able to facilitate the assembly of said rigid block (15; 115).

33- Device as in claim 32, characterized in that said assembly means (36; 136a, 136b) comprise guide elements (39; 43, 44) able to cooperate with mating holes (28, 31, 35) made respectively in said elements (18, 12, 18b; 118, 112, 120).

34- Device as in any claims from 24 to 33 inclusive, characterized in that it comprises automatic distribution means for said samples of liquid containing biomolecules in solution inside said holes (11) made on said supports (12; 112).

35- Device as in claim 34, characterized in that said automatic distribution means comprise at least plate means (52) able to move at least on a plane parallel to the plane on which said rigid block (15; 115) lies, said plate means (52) including through holes (56) able to allow said samples of liquid to pass.

36- Device as in claim 35, characterized in that said through holes (56) have a substantially conical conformation.

37- Device as in claim 35, characterized in that said plate means (52) are associated with cylinder-type movement means (53) and thrust means (55) to transmit motion.

38- Device as in claim 35, characterized in that said movable plate means (52) and said rigid block (15; 115) comprising said supports (12) are equipped with a reciprocal vertical movement to be taken into reciprocal contact at least during the step of unloading the samples of liquid from the holes (56) of said movable plate (52) to the holes (11) of said supports (12).

39- Device as in claim 38, characterized in that at least said rigid block (15; 115) is mounted on a plane (47) movable at least in height.

40- Device as in claim 35, characterized in that it comprises washing means (57) able to perform a washing cycle to wash the holes (56) of said movable plate means (52) after every cycle to distribute said samples of liquid.

41- Device as in claim 35, characterized in that the number of holes (56) on said movable plate means (52) is equal to a sub-multiple of the number of holes (11) on said supports (12).
